(19) United States
(12) Patent Application Publication
(10) Pub. No.: US 2024/0197793 A1
(43) Pub. Date: Jun. 20, 2024

(54) Antimicrobial and Antiviral Agent, Antimicrobial and Antiviral Member, and Method for Producing Antimicrobial and Antiviral Agent

(71) Applicant: Murata Manufacturing Co., Ltd., Nagaokakyo-shi (JP)

(72) Inventors: Masachika TAKATA, Nagaokakyo-shi (JP); Hirofumi SUNAHARA, Nagaokakyo-shi (JP)

(73) Assignee: Murata Manufacturing Co., Ltd., Nagaokakyo-shi (JP)

(21) Appl. No.: 18/593,410

(22) Filed: Mar. 1, 2024

Related U.S. Application Data
(63) Continuation of application No. 17/468,765, filed on Sep. 8, 2021, now Pat. No. 11,944,651.
Application No. 17/468,765 is a continuation of application No. 16/218,763, filed on Dec. 13, 2018, now abandoned.
Application No. 16/218,763 is a continuation of application No. PCT/JP2017/021563, filed on Jun. 12, 2017.

(30) Foreign Application Priority Data
Jun. 13, 2016 (JP) 2016-117443

(51) Int. Cl. A61K 35/74; A61K 9/06; A61K 35/747; A61K 36/232; A61K 36/282; A61P 31/04; A61P 31/10; A61P 31/12; A61P 31/16; C12N 1/04; C12N 1/20

(52) U.S. Cl. CPC A61K 35/74; A61K 9/06; A61K 35/747; A61K 36/232; A61K 36/282; A61P 31/04; A61P 31/10; A61P 31/12; A61P 31/16; C12N 1/04; C12N 1/20

(57) ABSTRACT

The present invention aims to provide an antimicrobial and antiviral agent which comprises a naturally occurring component as an active ingredient and is effective against microbes including various classes of fungi and viruses. In one aspect of the present invention, provided is an antimicrobial and antiviral agent comprising Lactobacillus derived from Artemisia indica var. maximowiczii.

### CROSS-REFERENCE TO RELATED APPLICATIONS

The following applications are incorporated herein by reference: PCT/JP2017/021563 (filed Jun. 12, 2017) and JP 2016-117443 (filed Jun. 13, 2016).

### BACKGROUND OF THE INVENTION

### Technical Field

The present invention relates to an antimicrobial and antiviral agent, an antimicrobial and antiviral member, and a method of manufacturing the antimicrobial and antiviral agent.

### Background Art

Some lactic acid bacteria are known to counteract pathogenic microbes harmful to humans and animals. For example, Patent Literature 1 discloses the use of Lactobacillus pentosus derived from Alpinia zerumbet for eradicating Helicobacter pylori. However, Patent Literature 1 discloses that a concentrated fermentation liquor of Alpinia zerumbet has no effect on candida.

Patent Literature 2 discloses that Lactobacillus reuteri produces an antimicrobial substance called reuterin and can inhibit proliferation of bacteria including salmonellae and some fungi. Patent Literature 2 also discloses the use of Lactobacillus fermentum for the treatment and prevention of skin disorders. Patent Literature 2 further discloses the use of Lactobacillus reuteri for the prevention or treatment of skin disorders caused by pathogenic microbes such as Staphylococcus aureus, Streptococcus pyogenes, and Propionibacterium acnes, and for the prevention or treatment of methicillin-resistant Staphylococcus aureus (MRSA) infection.

Patent Literature 3 discloses that Lactobacillus plantarum can suppress proliferation of Gram-negative bacteria such as Escherichia coli, Klebsiella pneumoniae, Pseudomonas aeruginosa, and Staphylococcus aureus and Gram-positive bacteria but cannot suppress proliferation of fungi such as Candida albicans and Candida parapsilosis.

Patent Literature 4 discloses that Lactobacillus casei KE01 can reduce Escherichia coli over several weeks. Patent Literature 5 discloses that Lactobacillus gasseri can reduce Salmonella enteritidis. Patent Literature 6 discloses that a biofilm consisting of yeast and Lactobacillus plantarum ML11-11 can reduce Bacillus subtilis.

Patent Literature 7 discloses that Lactobacillus pentosus can suppress proliferation of candida. However, Patent Literature 7 does not disclose that Lactobacillus pentosus can reduce candida.

### CITATION LIST

### Patent Literature

Patent Literature 1: Japanese Patent Laid-Open No. 2015-193619
Patent Literature 2: Japanese Patent Laid-Open No. 2014-230541
Patent Literature 3: Japanese Patent Laid-Open No. 2010-215641
Patent Literature 4: Japanese Patent Laid-Open No. 2010-195778
Patent Literature 5: Japanese Patent Laid-Open No. 2012-147759
Patent Literature 6: Japanese Patent Laid-Open No. 2013-150598
Patent Literature 7: Japanese Patent Laid-Open No. 2007-308504

### BRIEF SUMMARY OF THE INVENTION

### Technical Problem

Although alcohols may be used as an antimicrobial agent, some bacteria that form spores, such as Bacillus cereus, Bacillus subtilis, and Bacillus subtilis var. natto, are alcohol-resistant. Alcohols can cause side effects such as a rash and skin irritation. In addition, antimicrobial agents comprising a synthetic compound as an active ingredient may irritate the affected areas and cause symptoms of contact dermatitis that itch and are painful. Therefore, it is desirable to develop an antimicrobial and antiviral agent that contains a naturally occurring component as an active ingredient and is effective against microbes including various classes of fungi and viruses. The present invention, which has been achieved in light of such circumstances, aims to provide an antimicrobial and antiviral agent that comprises a naturally occurring component as an active ingredient and is effective against microbes including various classes of fungi and viruses, an antimicrobial and antiviral member, and a method of manufacturing the antimicrobial and antiviral agent.

### Solution to Problem

The antimicrobial and antiviral agent according to one aspect of the present invention includes Lactobacillus derived from Artemisia indica var. maximowiczii. The antimicrobial and antiviral agent according to one aspect of the present invention also includes a secretion from Lactobacillus derived from Artemisia indica var. maximowiczii. Further, the antimicrobial and antiviral agent according to one aspect of the present invention includes Lactobacillus, wherein the Lactobacillus is at least one species selected from the group consisting of parafarraginis, parabuchneri, buchneri, harbinensis, vini, and nagelii. Further, the antimicrobial and antiviral agent according to one aspect of the present invention also includes a secretion from Lactobacillus, wherein the Lactobacillus is at least one species selected from the group consisting of parafarraginis, parabuchneri, buchneri, harbinensis, vini, and nagelii. Furthermore, the antimicrobial and antiviral member according to one aspect of the present invention includes a member and the antimicrobial and antiviral agent disposed on a surface of the member. A method of manufacturing the antimicrobial and antiviral agent according to one aspect of the present invention includes fermenting a plant to provide a fermentation liquor containing Lactobacillus.

### Advantageous Effects of Invention

The present invention provides an antimicrobial and antiviral agent that comprises a naturally occurring component as an active ingredient and is effective against microbes including various classes of fungi and viruses, an antimicrobial and antiviral member, and a method of manufacturing the antimicrobial and antiviral agent.

### BRIEF DESCRIPTION OF THE DRAWINGS

FIG. 1 is a graph and table showing results of analyzing bacteria contained in the fermentation liquor according to Example 1.

FIG. 2 is a graph and table showing results of analyzing bacteria contained in the fermentation liquor according to Example 2.

FIG. 3 is a graph and table showing antimicrobial effect of the fermentation liquor according to Example 4.

FIG. 4 is a graph and table showing antifungal effect of the fermentation liquor according to Example 5.

FIG. 5 is a graph and table showing antiviral effect of the fermentation liquor according to Example 6.

FIG. 6 is a graph and table showing antiviral effect of the fermentation liquor according to Example 6.

FIG. 7 is a graph and table showing anti-Trichophyton effect of the fermentation liquor according to Example 7.

### DESCRIPTION OF EMBODIMENTS

Embodiments of the present invention will be now described in detail. It should be understood that embodiments as described below illustrate a device or method for embodying technical idea of the present invention and thus the technical idea of the present invention does not limit combination of constituent members to those as described below. The technical idea of the present invention may make various changes within the scope of the claims.

### First Embodiment

The antimicrobial and antiviral agent according to the first embodiment of the present invention includes at least one selected from the group consisting of Lactobacillus and a secretion from Lactobacillus. Lactobacillus is a species of lactobacilli and is a Gram-positive facultative anaerobe. Lactobacillus ferments sugar to produce lactic acid. Although Lactobacillus inhabits the living bodies of animals including human, Lactobacillus derived from plants is preferable for the Lactobacillus according to the first embodiment.

For example, the Lactobacillus according to the first embodiment can be extracted from fermented plants. Examples of the plants include, but not limited to, Artemisia indica var. maximowiczii, Angelica keiskei, Isodonis herba, and Theobroma cacao. Examples of Lactobacillus derived from Artemisia indica var. maximowiczii include Lactobacillus parafarraginis, Lactobacillus parabuchneri, Lactobacillus buchneri, and Lactobacillus harbinensis. Examples of Lactobacillus derived from Angelica keiskei include Lactobacillus vini and Lactobacillus nagelii. The antimicrobial and antiviral agent according to the first embodiment may include a plurality of Lactobacillus species.

The antimicrobial and antiviral agent according to the first embodiment can reduce the number of fungi (molds). The antimicrobial and antiviral agent according to the first embodiment can reduce for example 80% or more, 85% or more, 90% or more, or 95% or more of the fungi within 24 hours. Examples of the fungi include, but not limited to, Trichophyton, Candida, Cryptococcus, and Aspergillus. The antimicrobial and antiviral agent according to the first embodiment can be used as a therapeutic agent for mycosis. The mycosis includes, but not limited to, trichophytosis, candidosis, cryptococcosis, and aspergillosis.

The antimicrobial and antiviral agent according to the first embodiment can reduce the number of Gram-negative and Gram-positive bacteria. The antimicrobial and antiviral agent according to the first embodiment can reduce for example 80% or more, 85% or more, 90% or more, or 95% or more of the Gram-negative and Gram-positive bacteria within 24 hours. The Gram-negative bacteria include, but not limited to, Escherichia coli, Salmonella enterica, Vibrio parahaemolyticus, Klebsiella pneumoniae, Pseudomonas aeruginosa, and the like. The Gram-positive bacteria include, but not limited to, methicillin-resistant Staphylococcus aureus (MRSA), Bacillus cereus which forms spores, Bacillus subtilis, and the like. The antimicrobial and antiviral agent according to the first embodiment can be used as a disinfectant against Gram-negative and Gram-positive bacteria.

The antimicrobial and antiviral agent according to the first embodiment can reduce the number of viruses. The antimicrobial and antiviral agent according to the first embodiment can reduce for example 80% or more, 85% or more, 90% or more, or 95% or more of the viruses within 24 hours. The viruses include enveloped viruses which are viruses with envelope and non-enveloped viruses which are viruses without envelope. The viruses also include DNA and RNA viruses.

DNA viruses with envelope include, but not limited to, human herpes virus, vaccinia virus, hepatitis B virus, and the like.

RNA viruses with envelope include, but not limited to, influenza virus, SARS coronavirus, RS virus, mumps virus, Lassa virus, dengue virus, rubella virus, human immunodeficiency virus, measles virus, hepatitis C virus, Ebola virus, yellow fever virus, Japanese encephalitis virus, and the like.

DNA viruses without envelope include, but not limited to, adenovirus, B19 virus, papovavirus, human papillomavirus, and the like.

RNA viruses without envelope include, but not limited to, norovirus, polioviruses, echovirus, hepatitis A virus, hepatitis E virus, rhinovirus, astrovirus, rotavirus, coxsackievirus, enterovirus, sapovirus, and the like.

The antimicrobial and antiviral agent according to the first embodiment can be used as a disinfectant against viruses.

The antimicrobial and antiviral agent according to the first embodiment includes an effective amount of Lactobacillus and/or an effective amount of a secretion from Lactobacillus. The effective amount refers to an amount necessary to exert an antimicrobial or antiviral effect and thus will be appropriately determined depending on microbes, viruses, and symptoms of interest. The antimicrobial and antiviral agent according to the first embodiment includes Lactobacillus, for example, in a concentration of 0.001 wt % or more, 0.005 wt % or more, or 0.01 wt % or more. The antimicrobial and antiviral agent according to the first embodiment also includes Lactobacillus, for example, in a concentration of 20 wt % or less, 15 wt % or less, or 10 wt % or less but may include Lactobacillus in a higher concentration. However, the viscosity tends to increase with increasing the concentration of Lactobacillus.

The Lactobacillus contained in the antimicrobial and antiviral agent according to the first embodiment may be a living bacterium or a bacterium killed, for example, by heat treatment. Therefore, the antimicrobial and antiviral agent according to the first embodiment may include a killed bacterium of Lactobacillus. The Lactobacillus may be a dried bacterial product. The killed bacterium or dried bacterial product of Lactobacillus also has an antimicrobial and antiviral effect. Moreover, the killed bacterium or dried bacterial product of Lactobacillus can be easily transported and stored over an extended period.

The antimicrobial and antiviral agent according to the first embodiment may be, for example, a liquid, a cream, an ointment, a plaster, a gel, a wax, or a spray. The antimicrobial and antiviral agent according to the first embodiment is also administered in, for example, a disinfectant, a skin external medicine including a therapeutic agent for application, an eye drop, and an internal medicine. The antimicrobial and antiviral agent according to the first embodiment can be applied to, for example, human skin including fingers and toes, hair, mouth cavity, eyeball, and the like. The antimicrobial and antiviral agent according to the first embodiment can also be applied to, for example, cooking devices, walls and floors of buildings such as hospitals, and furniture such as a desk.

The antimicrobial and antiviral agent according to the first embodiment may appropriately include, in addition to Lactobacillus, components blended for cosmetics and pharmaceuticals depending on its purpose such as a liquid fat, a solid fat, a wax, a hydrocarbon, a higher fatty acid, a higher alcohol, an ester, a silicone, an anionic surfactant, a cationic surfactant, an amphoteric surfactant, a nonionic surfactant, a humectant, a water-soluble polymer, a thickening agent, a film-forming agent, a sequestering agent, a lower alcohol, a polyhydric alcohol, saccharides, amino acids, organic amines, a pH adjusting agent, skin nutrients, vitamins, an antioxidant, a flavor, a powder, a coloring material, and water.

The antimicrobial and antiviral agent according to the first embodiment may appropriately include depending on its purpose, in addition to Lactobacillus, an antimicrobial substance or an antiviral substance.

Lactobacillus and a secretion from Lactobacillus to be used as an active ingredient in the antimicrobial and antiviral agent according to the first embodiment can reduce microbes including various classes of fungi and viruses causing various diseases such as food poisoning. Lactobacillus and a secretion from Lactobacillus to be used as an active ingredient in the antimicrobial and antiviral agent according to the first embodiment can also reduce Bacillus cereus which forms alcohol-resistant spores.

Furthermore, Lactobacillus, which is a naturally occurring lactic acid bacterium, to be used as an active ingredient in the antimicrobial and antiviral agent according to the first embodiment has no or little deleterious effects including side effects and thus is safe when administered to humans. Also, for example the antimicrobial and antiviral agent according to the first embodiment to be given as a skin external medicine has no or little deleterious effects including side effects in the event of accidental ingestion by infants, children, or elderly persons because Lactobacillus is an edible lactic acid bacterium and can be easily degraded by digestive enzymes in the intestinal tract. The same holds true for the secretion of Lactobacillus.

The antimicrobial and antiviral agent according to the first embodiment is produced by fermenting a plant to provide a fermentation liquor containing Lactobacillus. When the plant is fermented, salts and sugars such as molasses are added to the plant. The fermentation is performed at a temperature of, for example, 30° C. The resulting fermentation liquor has a hydrogen ion exponent (pH) of about 4.0. A secretion from Lactobacillus may be extracted from the fermentation liquor.

The resulting fermentation liquor may be heated to kill Lactobacillus contained in the fermentation liquor. The fermentation liquor may also be spray dried to provide a dried bacterial product of Lactobacillus. The dried bacterial product can be prepared by freeze-drying (lyophilization), hot-air drying, or the like.

Furthermore, the resulting fermentation liquor, bacterium of Lactobacillus, or dried bacterial product of Lactobacillus may be added to soymilk which may be fermented to provide a soymilk fermentation liquor. The soymilk fermentation liquor also has an antimicrobial and antiviral effect.

### Second Embodiment

In the second and subsequent embodiments, description of similarity to the first embodiment is omitted and only points differing from the first embodiment will be described. In particular, similar actions and effects through a similar configuration are not mentioned in detail in each embodiment.

The antimicrobial and antiviral member according to the second embodiment includes a member and the antimicrobial and antiviral agent according to the first embodiment disposed on a surface of the member. The member is solid and is made of a material including, but not limited to, for example, metal, resin, glass, ceramic, and wood. For example, when the antimicrobial and antiviral member according to the second embodiment is produced, the antimicrobial and antiviral agent according to the first embodiment is blended into a paint, a dye, a pigment, various resins, a synthetic rubber, a latex, a film, a fiber, or the like and the material having the antimicrobial and antiviral agent blended is applied, disposed, or laminated onto a surface of the member.

Examples of the concentration of the antimicrobial and antiviral agent blended in the material may include, but not limited to, 0.001 wt % or more, 0.005 wt % or more, or 0.01 wt % or more and 20 wt % or less, 15 wt % or less, or 10 wt % or less.

### EXAMPLES

Embodiments of the present invention will be described below. However, the present invention is of course not limited to the following Examples.

### Example 1

Lactobacillus Derived from Artemisia indica var. maximowiczii

It appears that in leaves of Artemisia indica var. maximowiczii, the number of lactic acid bacteria reaches the maximum for an hour before and after sunrise, i.e., a total of 2 hours throughout the day. It also appears that lactic acid bacteria will decrease and photosynthetic bacteria will increase except for this time period. Accordingly, the tip of Artemisia indica var. maximowiczii leaf of about 20 cm long was taken during those 2 hours described above. The first pickle barrel with inside covered with a plastic bag was immediately charged with 6.3 kg of taken Artemisia indica var. maximowiczii leaves and 3.2 kg of molasses and 0.6 kg of crude salt were sprinkled on the Artemisia indica var. maximowiczii leaves before closing the plastic bag to seal. A weight was placed on the plastic bag to pickle the Artemisia indica var. maximowiczii leaves.

Several days after the level of the pickle juice exceeded the level of Artemisia indica var. maximowiczii leaves, the weight was removed. Next, the second pickle barrel was charged with 10 L of chlorine-free water for rinse and the pickled Artemisia indica var. maximowiczii leaves and 10 kg of the pickle juice were added to the water. Additionally, the third pickle barrel was provided and a wire gauze filter was placed on the opening of the third pickle barrel. The pickled Artemisia indica var. maximowiczii leaves were taken from the second pickle barrel in small portions while being kneaded and washed by hand. The Artemisia indica var. maximowiczii leaves were squeezed by lightly pressing them with the palm to the wire gauze filter on the opening of the third pickle barrel to obtain pickle juice.

After all the Artemisia indica var. maximowiczii leaves were squeezed, the pickle juice left in the second pickle juice was filtered through the wire gauze filter. Next, molasses (Hateruma brown sugar) and crude salt were added to final concentrations of 10 wt % and 3 wt % respectively to the pickle juice in the third pickle barrel and dissolved in the pickle juice. The ambient temperature of the third pickle barrel was then increased to about 30° C. to start fermentation. At first, effervescence was observed with large bubbles which were then gradually changed to small bubbles, and finally the effervescence subsided. About 1 week after the effervescence subsided, pH was near 3.8. The pickle juice at that time was used as an Artemisia indica var. maximowiczii fermentation liquor. A portion of the resulting Artemisia indica var. maximowiczii fermentation liquor was heated at 70° C. for 30 minutes to kill bacteria and provide a heat-treated Artemisia indica var. maximowiczii fermentation liquor.

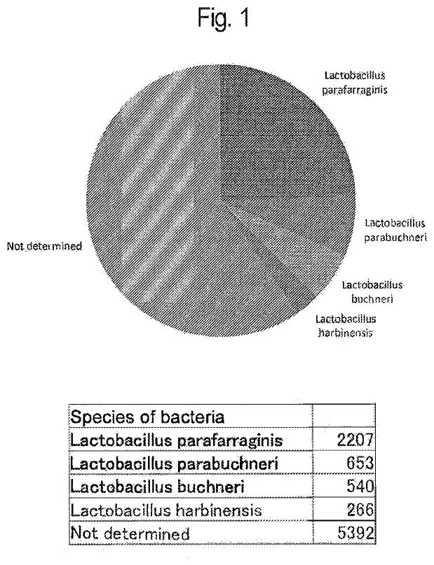

A non-heat-treated Artemisia indica var. maximowiczii fermentation liquor was analyzed with a next generation sequencer (MiSeq, Illumina, Inc.), and the Artemisia indica var. maximowiczii fermentation liquor contained Lactobacillus parafarraginis, Lactobacillus parabuchneri, Lactobacillus buchneri, Lactobacillus harbinensis, and the like, as shown in FIG. 1. It is noted that the next generation sequencer may be called high-throughput sequencer. Numerical values in the table in FIG. 1 represent the number of bacterial species contained in the Artemisia indica var. maximowiczii fermentation liquor.

### Example 2

Lactobacillus Derived from Angelica keiskei

It appears that in Angelica keiskei, the number of lactic acid bacteria reaches the maximum for an hour before and after sunrise, i.e., a total of 2 hours throughout the day. It also appears that lactic acid bacteria will decrease and photosynthetic bacteria will increase except for this time period. Accordingly, leaves and stems of Angelica keiskei sprouts were taken during the 2 hours. The first pickle barrel with inside covered with a plastic bag was immediately charged with 6.3 kg of taken Angelica keiskei and 3.2 kg of molasses and 0.6 kg of crude salt were sprinkled on the Angelica keiskei before closing the plastic bag to seal. A weight was placed on the plastic bag to pickle Angelica keiskei.

Several days after the level of the pickle juice exceeded the level of Angelica keiskei, the weight was removed. Next, the second pickle barrel was charged with 10 L of chlorine-free water for rinse and the pickled Angelica keiskei and 10 kg of the pickle juice were added to the water. Additionally, the third pickle barrel was provided and a wire gauze filter was placed on the opening of the third pickle barrel. The pickled Angelica keiskei was taken from the second pickle barrel in small portions while being kneaded and washed by hand. The Angelica keiskei was squeezed by lightly pressing it with the palm to the wire gauze filter on the opening of the third pickle barrel to obtain pickle juice.

After all the Angelica keiskei was squeezed, the pickle juice left in the second pickle juice was filtered through the wire gauze filter. Next, molasses and crude salt were added to final concentrations of 10 wt % and 3 wt % respectively to the pickle juice in the third pickle barrel and dissolved in the pickle juice. The ambient temperature of the third pickle barrel was then increased to about 30° C. to start fermentation. At first, effervescence was observed with large bubbles which were then gradually changed to small bubbles, and finally the effervescence subsided. About 1 week after the effervescence subsided, pH was near 4.0. The pickle juice at that time was used as an Angelica keiskei fermentation liquor. A portion of the resulting Angelica keiskei fermentation liquor was heated at 70° C. for 30 minutes to kill bacteria and provide a heat-treated Angelica keiskei fermentation liquor.

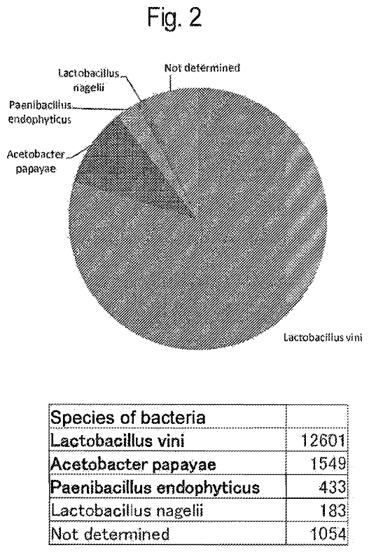

A non-heat-treated Angelica keiskei fermentation liquor was analyzed with a next generation sequencer (MiSeq, Illumina, Inc.), and the Angelica keiskei fermentation liquor contained species vini, nagelii, and the like, as shown in FIG. 2. It is noted that numerical values in the table in FIG. 2 represent the number of bacterial species contained in the Angelica keiskei fermentation liquor.

### Example 3

Soymilk Fermentation Liquor Prepared with Lactobacillus

Soymilk was heat-sterilized for about 30 minutes by heating it to 70° C. To the heat-sterilized soymilk was added the non-heat-treated Artemisia indica var. maximowiczii fermentation liquor prepared in Example 1 to a final concentration of about 10 wt % and stirred thoroughly. Soymilk containing the non-heat-treated Artemisia indica var. maximowiczii fermentation liquor was then fermented at 37ºC for 24 hours. After fermentation, solid substances were removed by filtration to provide a soymilk fermentation liquor containing Lactobacillus.

### Example 4

### Antimicrobial Effect of Lactobacillus

Staphylococcus aureus and MRSA were provided as Gram-positive cocci; Bacillus subtilis and Bacillus cereus were provided as Gram-positive bacilli; Escherichia coli, Salmonella enterica, Vibrio parahaemolyticus, and Klebsiella pneumoniae were provided as Gram-negative cocci; and Pseudomonas aeruginosa was provided as a Gram-negative bacillus.

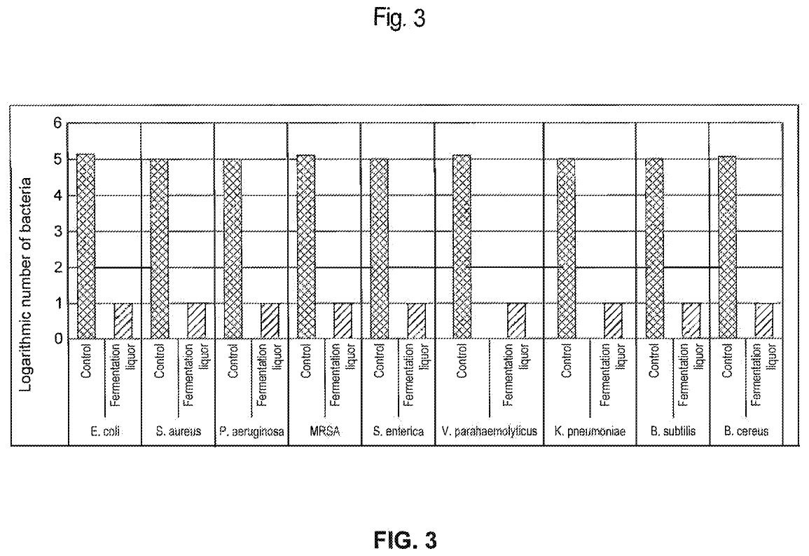

10 mL of the soymilk fermentation liquor containing Lactobacillus prepared in Example 1 was inoculated with 0.1 mL of a bacterial suspension containing any of the bacteria as described above in a concentration of 107/mL and allowed to act at 25° C. The viable cell count of the inoculated bacterium was measured over time for 24 hours. As a control, 10 mL of 1/15 mol/L in a concentration of phosphate buffer, pH 7.2 was inoculated with 0.1 mL of the bacterial suspension and allowed to act at 25° C. The viable cell count of the inoculated bacterium was measured over time for 24 hours. As a result, as shown in FIG. 3, the soymilk fermentation liquor containing Lactobacillus decreased all the species of bacteria provided within 24 hours.

### Example 5

### Antifungal Effect of Lactobacillus

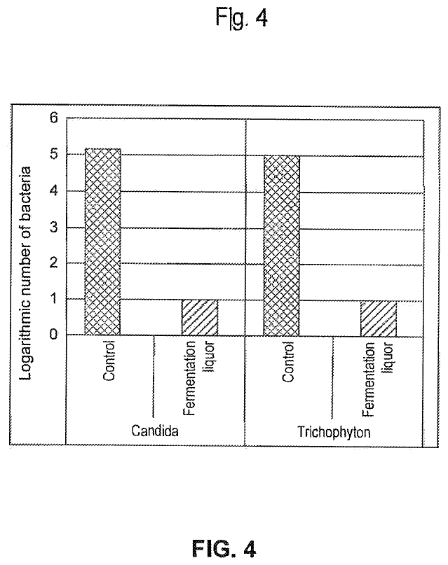

Trichophyton and Candida were provided as fungi. 10 mL of the soymilk fermentation liquor containing Lactobacillus prepared in Example 1 was inoculated with 0.1 mL of a fungal suspension containing Trichophyton or Candida in a concentration of 10⁷/mL and allowed to act at 25° C. The viable cell count of the inoculated fungus was measured over time for 24 hours. As a control, 10 mL of 1/15 mol/L in a concentration of a phosphate buffer, pH 7.2 was inoculated with 0.1 mL of the fungal suspension and allowed to act at 25° C. The viable cell count of the inoculated fungus was measured over time for 24 hours. As a result, as shown in FIG. 4, the soymilk fermentation liquor containing Lactobacillus decreased the Trichophyton and Candida provided within 24 hours.

### Example 6

### Antiviral Effect of Lactobacillus

The culture supernatant containing influenza virus A (H1N1) was provided as an enveloped virus. The culture supernatant containing norovirus (feline calicivirus) was also provided as a non-enveloped virus. These virus-containing culture supernatants were 10-fold serially diluted with purified water. Subsequently, an antiviral test was performed at room temperature to determine 50% Tissue Culture Infectious Dose (TCID50). The antiviral test was performed at Japan Food Research Laboratories.

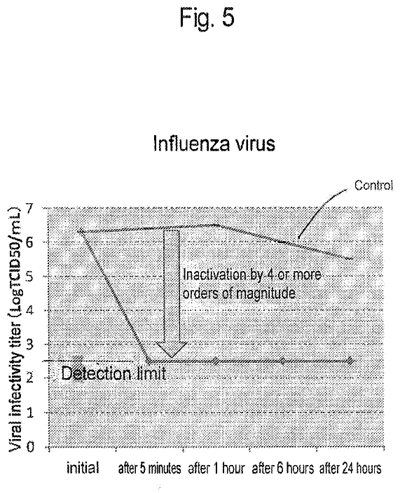

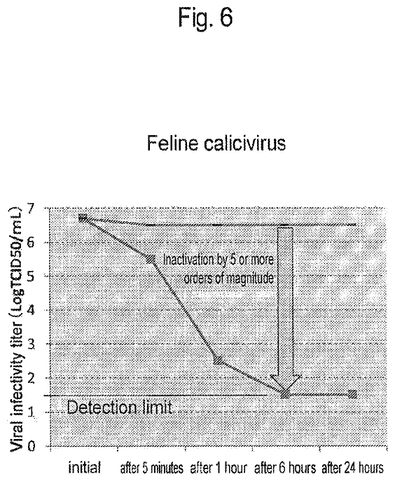

As a result, as shown in FIG. 5, the soymilk fermentation liquor containing Lactobacillus decreased the infectivity titer of influenza virus within 1 hour. As shown in FIG. 6, the soymilk fermentation liquor containing Lactobacillus also decreased the infectivity titer of norovirus within 24 hours.

### Example 7

### Anti-Trichophyton Effect of Lactobacillus

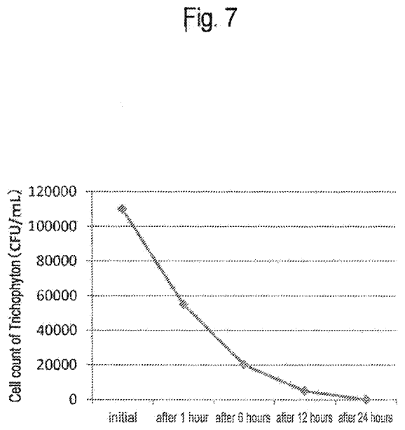

The non-heat-treated Artemisia indica var. maximowiczii fermentation liquor prepared in Example 1 was spray-dried to provide a dried fungus of Lactobacillus. The dried fungus was suspended in water and glycerin to a concentration of 10 parts by weight to provide a Lactobacillus suspension according to Example 7. The Lactobacillus suspension was added to Trichophyton and colony-forming unit (CFU) of Trichophyton was measured, which revealed that, as shown in FIG. 7, the Lactobacillus suspension killed Trichophyton within 24 hours.

Each of the embodiments and Examples as described above is intended to facilitate understanding of the present invention but not to limit the interpretation of the present invention. The present invention may be altered or modified without departing from the spirit thereof and includes equivalents thereof. That is to say, the design of each of the embodiments and Examples appropriately changed by those skilled in the art is within the scope of the present invention as long as it includes a feature of the present invention. For example, each element comprised in each of the embodiments and Examples is not limited to the exemplified elements and may be appropriately changed. Any of the embodiments and Examples is also exemplary and thus, needless to say, components described in other embodiments may be partially substituted or combined. These partial substitution and combination are also within the scope of the present invention as long as they include a feature of the present invention.

1.-21. (canceled)

22. A method for reducing microbial disease comprising administering an antimicrobial agent to a subject, the agent comprising Lactobacillus derived from Artemisia indica var. maximowiczii or a secretion from the Lactobacillus derived from Artemisia indica var. maximowiczii, wherein the agent is a fermentation product of Artemisia indica var. maximowiczii.

23. The method according to claim 22, wherein the agent is a lipid, a cream, an ointment, a plaster, a gel, a wax, or a spray.

24. The method according to claim 22, wherein the Lactobacillus is at least one species selected from the group consisting of parafarraginis, parabuchneri, buchneri, and harbinensis.
